SEQUENCE LISTING (4 sequences)

SEQ ID NO: 1 - protein, 33 aa, synthetic construct
  REGION 1..33: note = Synthetic
FNMQQQRRFYEALHDPNLNEEQRNAKIKSIRDD

SEQ ID NO: 2 - skipped

SEQ ID NO: 3 - protein, 4 aa, synthetic construct
  REGION 1..4: note = Synthetic
VVEE

SEQ ID NO: 4 - protein, 4 aa, synthetic construct
  REGION 1..4: note = Synthetic
VVKK

(19) United States
(12) Patent Application Publication
(10) Pub. No.: US 2024/0327455 A1
(43) Pub. Date: Oct. 3, 2024

(54) HIGH-AFFINITY SUPRAMOLECULAR POLYMERS FOR BINDING-TRIGGERED ANTIBODY PRECIPITATION AND PURIFICATION

(71) Applicants: The Johns Hopkins University, Baltimore, MD (US); BRISTOL-MYERS SQUIBB COMPANY, Princeton, NJ (US)

(72) Inventors: Honggang Cui, Baltimore, MD (US); Yi Li, Baltimore, MD (US); David Stern, Baltimore, MD (US); XuanKuo Xu, Princeton, NJ (US); Lye Lin Lock, Princeton, NJ (US); Jason Mills, Princeton, NJ (US); Sanchaylta Ghose, Princeton, NJ (US); Zheng Jian Li, Princeton, NJ (US)

(21) Appl. No.: 18/579,578

(22) PCT Filed: Jul. 18, 2022
(86) PCT No.: PCT/US22/73822

§ 371 Date: Jan. 16, 2024

Related U.S. Application Data
(60) Provisional application No. 63/223,792, filed on Jul. 20, 2021.

(51) Int. Cl. C07K 1/36; C07K 1/107; C07K 1/32; C07K 14/315

(52) U.S. Cl. CPC C07K 1/36; C07K 1/1077; C07K 1/32; C07K 14/315

(57) ABSTRACT

The disclosure is directed to systems and methods for purifying proteins containing an Fc region, such as antibodies or Fc fusion proteins. The system includes one or more filler molecules comprising a linear hydrocarbon chain conjugated to a peptide amino acid sequence, and one or more ligand molecules comprising a linear hydrocarbon chain, a peptide amino acid sequence, a linker, and a Z33 peptide of Staphylococcus aureus Protein A. The filler molecules and ligand molecules self-assemble into immunofibers (IFs) under physiological conditions.

This application claims priority to U.S. provisional patent application Ser. No. 63/223,792, filed Jul. 20, 2021, which is incorporated herein by reference in its entirety.

### INCORPORATION-BY-REFERENCE OF MATERIAL SUBMITTED ELECTRONICALLY

The text of the computer readable sequence listing filed herewith, titled “JHU-39610-601_SQL”, created Jul. 18, 2022, having a file size of 5,022 bytes, is hereby incorporated by reference in its entirety.

### BACKGROUND

Supramolecular assembly is a bottom-up approach to construct hierarchical and functional nanostructures. Among the recently developed supramolecular polymers, peptide-based materials are notably appealing because of their biocompatibility, biodegradability, and low toxicity. The presentation of bioactive epitopes on a supramolecular substrate enables the active-targeting of the nanostructures for efficient molecular/cellular recognition and signaling, as well as the precise delivery and accumulation of therapeutics at desired sites. In light of the steric hindrance introduced by anchoring these epitopes on supramolecular polymer surfaces, the epitope spatial arrangement plays an important role in regulating their functionality. Co-assembly of bioactive building units with an inert molecule has been shown to effectively modulate the epitope density and achieve enhanced bioactivities. Meanwhile, the use of flexible linkers for spacing out the epitope in the radial direction of the resultant nanostructures is equally essential to improve the epitope accessibility and their interactions with target biomolecules.

Affinity precipitation has been increasingly explored as a promising alternative to protein A chromatography for the purification of monoclonal antibodies (mAbs) and other therapeutic proteins, as the conventional protein A-based affinity chromatography method suffers from limited production capacities and high media cost. The use of salt to trigger or aid in the precipitation of antibodies is prevalent during the purification process. However, high salt concentration is known to increase the risks of protein instability and the non-specific precipitation of impurities present in the solution.

There remains a need for more efficient systems and methods for capturing and purifying antibodies and other proteins.

### BRIEF SUMMARY OF THE DISCLOSURE

The disclosure provides a system comprising: (a) one or more filler molecules comprising a linear hydrocarbon chain conjugated to an amino acid sequence of 2-5 amino acids; (b) one or more ligand molecules comprising a linear hydrocarbon chain, an amino acid sequence of 2-5 amino acids, a linker, and a Z33 peptide of Staphylococcus aureus Protein A, or an antibody-binding fragment thereof, conjugated to the linker, wherein the one or more filler molecules and one or more ligand molecules have the ability to self-assemble into immunofibers (IFs) under physiological conditions. Also provided are methods of purifying an antibody and/or fusion Fc protein using the aforementioned system.

### BRIEF DESCRIPTION OF THE DRAWING(S)

FIGS. 1A-1C include schematic diagrams illustrating the molecular design of the immunofiber (IF) building units with OEG (or PEG) linkers and the formation of IFs for monoclonal antibody (mAb) capture. FIG. 1A shows the chemical structures of the filler molecule, C12-VVEE, and the ligand molecules, C12-VVKK[Linker]Z33. FIG. 1B is a schematic illustrating the ligand molecule design with various linkers and the effect of linker length on the presentation of Z33 peptide on co-assembled IFs. FIG. 1C illustrates co-assembly of filler and ligand molecules into supramolecular IFs and four possible states of mAbs in an IF solution: (1) two Fc-portions of a mAb bound to two ligands from two different IFs: (2) two Fc-portions of a mAb bound to two adjacent ligands from the same IF; (3) one Fc-portion of a mAb bound to one ligand on an IF: (4) free mAbs in the solution bound to zero, one, or two monomer ligands that are not assembled into IFs.

FIGS. 2A-2H show the characterization of filler and ligand molecules. FIGS. 2A-2G are representative TEM images of self-assembled filler and ligand molecules (2A: C12-VVEE, 2B:O4; 2C:O8; 2D:O12; 2E:O16; 2F:O36; and 2G: P2000) in PBS with a diameter of 7.4±0.6 nm, 11.1±0.9 nm, 12.6±1.2 nm, 13.6±1.2 nm, 14.9±1.3 nm, 18.5±1.8 nm, and 19.3±2.0 nm, respectively. Diameters are given as mean±SD (n=30). Scale bars: 100 nm. FIG. 2H is a graph illustrating normalized CD spectra of self-assembled ligand molecules with different OEG (or PEG) linkers, suggesting the formation and retention of α-helix secondary structures.

FIGS. 3A-3D are graphs illustrating ITC profiles and binding curves for the stepwise injection of 100 μM mAb1 into 40 μM O4 (3A), O8 (3B), O12 (3C), O16 (3D) at 25° C. in PBS, pH 7.4.

FIGS. 4A-4E show comparisons of mAb precipitation performance of IFs with different ligand molecules. FIGS. 4A and 4B are graphs illustrating mAb precipitation yields for 20 μM and 40 μM mAb1, respectively, incubated with various co-assembled IFs (2.5 mM filler, 100 μM ligand) under 1 M salt or no salt conditions. The data point for G2 with 1 M salt in FIG. 4A was replotted from reference. FIGS. 4C and 4D are graphs illustrating precipitated mAb mass from 100 μL samples of 20 μM (0.3 mg) (4C) and 40 μM (0.6 mg) (4D) mAb1 incubated with various 100 IFs (2.5 mM filler, 100 μM ligand) under 1 M salt or no salt conditions. FIG. 4E includes photographs of co-assembled IFs (2.5 mM filler, 100 μM ligand) after mixing with 40 μM mAb for 5 minutes under 1 M salt or no salt conditions. All experiments were performed in triplicate and the data is shown as mean±standard deviation.

FIGS. 5A-5F show optimization of mAb precipitation yield. FIGS. 5A and 5B are graphs illustrating the yield and mass, respectively, of mAb precipitation for 100 μL mAb1 at 40 μM (0.6 mg), 80 μM (1.2 mg), and 133 μM (2 mg) incubated with IFs at various O16 concentrations. FIG. 5C is a graph illustrating a comparison of mAb precipitation yield for 40 μM mAb1 incubated with optimized IFs (2.5 mM filler, 250 μM O16) under 1 M salt and no salt conditions. FIG. 5D is a schematic diagram showing a proposed mechanism of mAb-IF agglomeration as the ligand concentration increases for a fixed mAb concentration. mAbs at States 3, State 1, and State 2 are dominant at low, medium, and high ligand concentrations, respectively. FIG. 5E is a graph showing turbidity of 40 μM mAb with optimized IFs (2.5 mM filler, 250 μM O16) in 2 hours. FIG. 5F is a graph showing the effect of binding time on the mAb precipitation yield for 40 μM mAb1 incubated with optimized IFs (2.5 mM filler, 250 μM O16). All experiments were performed in triplicate and the data is shown as mean±standard deviation.

FIGS. 6A-6F show purification of mAb from clarified bulk cell culture harvest (abbreviated as “mAb CB”) and separation of mAbs at high titers. FIG. 6A is a schematic illustration of the mAb purification process using IFs in PBS solution. FIG. 6B is a graph showing mAb precipitation yield, yield loss at wash step, and elution yield for 40 μM and 80 μM pure mAb1 after two sequential precipitations under no salt conditions. FIG. 6C is a graph showing mAb precipitation yields for 40 μM and 80 μM mAb1 CB after two sequential precipitations with IF solutions under no salt conditions. FIG. 6D is a schematic illustration of the high titer mAb purification process using solid IFs in lyophilized powder form. FIGS. 6E and 6F are graphs showing precipitation yields for 21 mg/mL (6E) and 31 mg/ml (6F) pure mAb1 using sequential precipitation with lyophilized IFs under no salt conditions. All experiments were performed in triplicate and the data is shown as mean±standard deviation.

FIG. 7 is a graph illustrating mAb precipitation yields for 100 μL mAb1 at 10 μM and 20 μM after an incubation with 100 μL IFs at various O16 concentrations. IFs with 200 μM O16 gave the best mAb precipitation yield for both mAb concentrations.

### Definitions

To facilitate an understanding of the present technology, a number of terms and phrases are defined below. Additional definitions are set forth throughout the detailed description.

The terms “nucleic acid,” “polynucleotide,” “nucleotide sequence,” and “oligonucleotide” are used interchangeably herein and refer to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The terms encompass any deoxyribonucleotide, ribonucleotide, or peptide nucleic acid component, and any chemical variants thereof, such as methylated, hydroxy methylated, or glycosylated forms of these bases. The polymers or oligomers may be heterogenous or homogenous in composition, may be isolated from naturally occurring sources, or may be artificially or synthetically produced. In addition, nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41 (14): 4503-4510 (2002) and U.S. Pat. No. 5,034,506), locked nucleic acid (LNA: see Wahlestedt et al., Proc. Natl. Acad. Sci. U.S.A., 97:5633-5638 (2000)), cyclohexenyl nucleic acids (see Wang, J. Am. Chem. Soc., 122:8595-8602 (2000)), and/or a ribozyme. The terms “nucleic acid” and “nucleic acid sequence” may also encompass a chain comprising non-natural nucleotides, modified nucleotides, and/or non-nucleotide building blocks that can exhibit the same function as natural nucleotides (e.g., “nucleotide analogs”).

The term “amino acid” refers to natural amino acids, unnatural amino acids, and amino acid analogs, all in their D and L stereoisomers, unless otherwise indicated, if their structures allow such stereoisomeric forms.

Natural amino acids include alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V).

Unnatural amino acids include, but are not limited to, azetidinecarboxylic acid, 2-aminoadipic acid, 3-aminoadipic acid, beta-alanine, naphthylalanine (“naph”), aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine (“tBuG”), 2,4-diaminoisobutyric acid, desmosine, 2,2′-diaminopimelic acid, 2,3-diaminopropionic acid, N-ethylglycine, N-ethylasparagine, homoproline (“hPro” or “homoP”), hydroxylysine, allo-hydroxylysine, 3-hydroxyproline (“3Hyp”), 4-hydroxyproline (“4Hyp”), isodesmosine, allo-isoleucine, N-methylalanine (“MeAla” or “Nime”), N-alkylglycine (“NAG”) including N-methylglycine, N-methylisoleucine, N-alkylpentylglycine (“NAPG”) including N-methylpentylglycine. N-methylvaline, naphthylalanine, norvaline (“Norval”), norleucine (“Norleu”), octylglycine (“OctG”), ornithine (“Orn”), pentylglycine (“pG” or “PGly.”), pipecolic acid, thioproline (“ThioP” or “tPro”), homoLysine (“hLys”), homoArginine (“hArg”), (S)—N-Fmoc-2-(4′-pentenyl) alanine, Fmoc-2,2-bis(4-pentenyl)glycine. Other unnatural amino acids that may be employed are disclosed in, e.g., International Patent Application Publication WO 2018/106937.

The terms “polypeptide” and “protein” are used interchangeably herein and refer to a polymeric form of amino acids of any length, which can include coded and non-coded amino acids, chemically or biochemically modified or derivatized amino acids, and polypeptides having modified peptide backbones.

The term, “peptide,” as used herein, includes a sequence of from four to 100 amino acid residues in length, preferably about 10 to 80 residues in length, more preferably, 15 to 65 residues in length, and in which the α-carboxyl group of one amino acid is joined by an amide bond to the main chain (α- or β-) amino group of the adjacent amino acid. A peptide may comprise natural amino acids, non-natural amino acids, amino acid analogs, and/or modified amino acids. A peptide may be a subsequence of naturally occurring protein or a non-natural (synthetic) sequence.

As used herein, the term “immuno-amphiphiles” means a molecule that can spontaneously associate into discrete, stable supramolecular nanostructures termed “immunofibers.” Generally, the IFs can assemble in a pH range between about 2.8 to about 7.5. However, the binding properties are also pH dependent. Those IFs which are more positively charged are easier to associate with higher pH solutions, and conversely, negatively charged IFs will associate easier in lower pH solutions.

As used herein, the term “antibody” refers to an immunoglobulin molecule that is typically composed of two identical pairs of polypeptide chains, each pair having one “light” (L) chain and one “heavy” (H) chain. Human light chains are classified as kappa and lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 3 or more amino acids. Each heavy chain is comprised of a heavy chain variable region (abbreviated herein as HCVR or V_{H}) and a heavy chain constant region. The heavy chain constant region is comprised of three domains, C_{H1}, C_{H2} and C_{H3}. Each light chain is comprised of a light chain variable region (abbreviated herein as LCVR or V_{L}) and a light chain constant region. The light chain constant region is comprised of one domain, C_{L}. The constant regions of the antibodies may mediate the binding of the immunoglobulin to host tissues or factors, including various cells of the immune system (e.g., effector cells) and the first component (C1q) of the classical complement system. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of each heavy/light chain pair (V_{H} and V_{L}), respectively, form the antibody binding site. The term “antibody” encompasses an antibody that is part of an antibody multimer (a multimeric form of antibodies), such as dimers, trimers, or higher-order multimers of monomeric antibodies. It also encompasses an antibody that is linked or attached to, or otherwise physically or functionally associated with, a non-antibody moiety. Further, the term “antibody” is not limited by any particular method of producing the antibody. For example, it includes, inter alia, recombinant antibodies, synthetic antibodies, monoclonal antibodies, polyclonal antibodies, bi-specific antibodies, and multi-specific antibodies.

The term “monoclonal antibody,” as used herein, refers to an antibody produced by a single clone of B lymphocytes that is directed against a single epitope on an antigen. Monoclonal antibodies typically are produced using hybridoma technology, as first described in Köhler and Milstein, Eur. J. Immunol., 5:511-519 (1976). Monoclonal antibodies may also be produced using recombinant DNA methods (see, e.g., U.S. Pat. No. 4,816,567), isolated from phage display antibody libraries (see, e.g., Clackson et al. Nature, 352:624-628 (1991)); and Marks et al., J. Mol. Biol., 222:581-597 (1991)), or produced from transgenic mice carrying a fully human immunoglobulin system (see, e.g., Lonberg, Nat. Biotechnol., 23 (9): 1117-25 (2005), and Lonberg, Handb. Exp. Pharmacol., 181:69-97 (2008)). In contrast, “polyclonal” antibodies are antibodies that are secreted by different B cell lineages within an animal. Polyclonal antibodies are a collection of immunoglobulin molecules that recognize multiple epitopes on the same antigen.

The terms “Fc region,” “Fc fragment,” or “fragment crystallizable region,” can be used interchangeably herein to refer to the region of a monoclonal antibody comprising the hinge and constant heavy-chain domains (CH2 and CH3). The Fc region mediates downstream effector functions via its interaction with Fc-receptors on (innate) immune cells or with C1q, the recognition molecule of the complement system. The interaction with Fc-receptors can exert a broad range of immunomodulatory functions, such as antibody-dependent cell-mediated cytotoxicity (ADCC) and antibody-dependent cellular phagocytosis (ADCP), in response to infectious agents.

The terms “fragment of an antibody,” “antibody fragment,” and “functional fragment of an antibody” are used interchangeably herein to mean one or more fragments of an antibody that retain the ability to specifically bind to an antigen (see, generally, Holliger et al., Nat. Biotech., 23 (9): 1126-1129 (2005)). An antibody fragment desirably comprises, for example, one or more CDRs, the variable region (or portions thereof), the constant region (or portions thereof), or combinations thereof. Examples of antibody fragments include, but are not limited to, (i) a Fab fragment, which is a monovalent fragment consisting of the V_{L}, V_{H}, C_{L}, and C_{H}1 domains, (ii) a F(a′)₂ fragment, which is a bivalent fragment comprising two Fab fragments linked by a disulfide bridge at the hinge region, (iii) a Fv fragment consisting of the V_{L} and V_{H} domains of a single arm of an antibody, (iv) a Fab′ fragment, which results from breaking the disulfide bridge of an F(ab′)₂ fragment using mild reducing conditions, (v) a disulfide-stabilized Fv fragment (dsFv), and (vi) a domain antibody (dAb), which is an antibody single variable region domain (V_{H} or V_{L}) polypeptide that specifically binds antigen.

As used herein, the term “antibody binding peptide” means a peptide that has the ability to bind an antibody, or a specific portion of an antibody molecule, for example, the Fc portion, with high specificity, such as having a K_{d} of between about 10⁻⁶ M to about 10⁻¹⁰ M.

As used herein, the term “sample” means any sample, solution, fluid, or mixture containing an antibody of interest or an Fc region-containing protein of interest (e.g., an Fc fusion protein) which can be bound using the immunofibers of the present invention. In some embodiments, the sample can be a biological sample. For example, the sample includes, for example, cell cultures, cell lysates, and/or clarified bulk (e.g., clarified cell culture supernatant). Optionally, the sample is produced from a host cell or organism that expresses the antibody or Fc region-containing protein of interest (either naturally or recombinantly). For example, the cells in a cell culture include host cells transfected with an expression construct containing a nucleic acid that encodes an antibody or Fc fusion protein of interest. These host cells can be bacterial cells, fungal cells, insect cells or, preferably, animal cells grown in culture. The terms “biological sample” and “biological fluid,” as used herein, refers to any quantity of a substance from a living or formerly living patient or mammal or from cultured cells. Such substances include, but are not limited to, blood, serum, plasma, urine, cells, organs, tissues, bone, bone marrow, lymph, lymph nodes, synovial tissue, chondrocytes, synovial macrophages, endothelial cells, skin, cell cultures, cell lysates, and clarified bulk (e.g., clarified cell culture supernatant).

The terms “monomer,” “monomeric subunit” and “monomeric unit” are used interchangeably herein and refer to one of the basic structural units of a polymer or oligomer. An “oligomer” is a molecule possessing from about 1 to about 30 monomers. The architecture of an oligomer can be, e.g., linear, branched, or forked. An oligomer is a type of polymer. The term “polymer,” as used herein, refers to a substance or material containing large molecules, or macromolecules, composed of many monomers (e.g., hundreds or thousands).

### DETAILED DESCRIPTION

The present disclosure is predicated, at least in part, on the construction of a supramolecular immunofiber (IF) system by co-assembling rationally-designed filler and ligand molecules for the affinity precipitation and purification of monoclonal antibodies (mAbs). In some embodiments, the ligand molecule comprises a protein A mimicking peptide that binds to the Fc-portion of immunoglobulin G (IgG) from most mammalian species in a pH-specific manner. In addition, a series of linkers may be incorporated into the ligand design to increase flexibility and accessibility of the protein A mimicking peptide. Given the dual Fc-binding sites on monoclonal antibodies, specifically IgG, and the multivalence of the immunofibers provided herein, the resulting enhanced multivalent mAb-IF binding could potentially result in IF crosslinking into large complexes and facilitate monoclonal antibody precipitation under low salt or no salt conditions.

In this regard, the disclosure provides a system comprising one or more filler molecules and one or more ligand molecules that have the ability to self-assemble into immunofibers (IFs) under physiological conditions. The terms “filler” or “filler molecule,” as used herein, refer to a molecule, particle, or compound that is added to a composition that can improve specific properties of the composition. In the context of the present disclosure, the one or more filler molecules are designed to modulate the distribution of the ligand molecule in the co-assembled immunofibers. The terms “ligand” or “ligand molecule,” as used herein, refer to a substance that forms a complex with a biomolecule to produce a biological effect. In some embodiments, a ligand produces a signal by binding to a site on a target protein (e.g., a receptor). Alternatively, a ligand may be a small molecule, ion, or protein which binds to a nucleic acid molecule, such as a DNA double helix. Other types of ligands include, but are not limited to, steroid hormones, growth factors, neurotransmitters, and other peptides.

In some embodiments, each of the one or more filler molecules and each of the one or more ligand molecules comprises a linear hydrocarbon chain conjugated to an amino acid sequence. It will be appreciated that a “hydrocarbon” is an organic compound consisting entirely of hydrogen and carbon. The linear hydrocarbon chain may be of any suitable length. In some embodiments, a linear hydrocarbon chain comprises from 1 to 24 carbon atoms, for example, 1 to 16 carbon atoms, 1 to 14 carbon atoms, 1 to 12 carbon atoms, 1 to 10 carbon atoms, 1 to 8 carbon atoms, 1 to 6 carbon atoms, or 1 to 4 carbon atoms. In some embodiments, the linear hydrocarbon chain comprises 10-15 carbon atoms (e.g., 10, 11, 12, 13, 14, or 15 carbon atoms). Ideally, the linear hydrocarbon chain comprises 12 carbon atoms. One of ordinary skill in the art will appreciate that there is an upper limit to the number of carbons included in the linear hydrocarbon chain to maintain solubility in an aqueous solution.

Each of the one or more filler molecules and each of the one or more ligand molecules also comprises an amino acid sequence conjugated to the linear hydrocarbon chain. The amino acid sequence present in the one or more filler molecules may be the same as or different than the amino acid sequence present in the one or more ligand molecules. Ideally, the amino acid sequences present in the filler and ligand molecules are designed to promote interaction among the molecules during co-assembly. The amino acid sequences may be of any suitable length. In some embodiments, the amino acid sequence present in a filler molecule and/or a ligand molecules comprises 1-20 amino acids, such as 1-5 amino acids, 5-10 amino acids, 1-10 amino acids, 10-15 amino acids, or 15-20 amino acids. For example, each of the one or more filler molecules comprises an amino acid sequence of 2-5 amino acids (e.g., 2, 3, 4, or 5 amino acids), and, in some aspects, 4 amino acids. Similarly, each of the one or more ligand molecules comprises an amino acid sequence of 2-5 amino acids (e.g., 2, 3, 4, or 5 amino acids), and, in some aspects, 4 amino acids. An exemplary amino acid sequence for inclusion in the one or more filler molecules and/or the one or more ligand molecules comprises VVXX (SEQ ID NO: 2, with “X” indicating any amino acid). In some embodiments, each of the one or more filler molecules comprises the amino acid sequence VVEE (SEQ ID NO: 3), and each of the one or more ligand molecules comprises the amino acid sequence VVKK (SEQ ID NO: 4). The disclosure is not limited to these particular amino acid sequences, however.

In addition to the linear hydrocarbon chain and the amino acid sequence, each of the one or more ligand molecules further comprises a linker and a Z33 peptide of Staphylococcus aureus Protein A, or an antibody-binding fragment thereof, conjugated to the linker. A “linker” is any chemical moiety that is capable of linking one compound to another compound (e.g., a cell-binding agent such as a peptide ligand or antibody) in a stable, covalent manner. Linkers can be susceptible to, or be substantially resistant to, acid-induced cleavage, light-induced cleavage, peptidase-induced cleavage, esterase-induced cleavage, and/or disulfide bond cleavage. In some embodiments, the linker can be any amino acid with a side chain having a free amino, carboxyl or disulfide group. Exemplary amino acids useful as amino acid linkers in the one or more filler molecules and/or one or more ligand molecules of the present invention include lysine (K), glutamic acid (E), arginine (R) and cysteine (C). Other suitable linkers are well known in the art and include, for example, disulfide groups, thioether groups, acid labile groups, photolabile groups, peptidase labile groups, and esterase labile groups. Linkers also include charged linkers and hydrophilic forms thereof as described herein and known in the art. In some embodiments, the linker may be a cleavable linker, a non-cleavable linker, a hydrophilic linker, and a dicarboxylic acid-based linker.

In some embodiments, the linker is a hydrophilic linker comprising one or more poly(ethylene glycol) (PEG) molecules or one or more oligo (ethylene glycol) (OEG) molecules. Unless otherwise indicated, a “PEG oligomer” or an oligoethylene glycol (OEG) is one in which all of the monomer subunits are ethylene oxide subunits. Typically, substantially all, or all, monomeric subunits are ethylene oxide subunits, though the oligomer may contain distinct end capping moieties or functional groups, e.g., for conjugation. Typically, PEG oligomers for use in the present disclosure will comprise one of the two following structures: “—(CH₂CH₂O)ₙ—” or “—(CH₂CH₂O)ₙ₋₁CH₂CH₂—,” depending upon whether or not the terminal oxygen(s) has been displaced, e.g., during a synthetic transformation. In some embodiments, the variable (n) ranges from 1 to 30, and the terminal groups and architecture of the overall PEG or OEG can vary.

PEGylation is a process through which polyethylene glycol chains are conjugated to proteins (e.g., therapeutic proteins), peptides, aptamers, enzymes, small molecule drugs, antibodies, and other molecules. Through the PEGylation process, the molecular mass of the conjugated protein is increased, resulting in reduced degradation and increased stability in vivo. PEGylation also reduces the immunogenicity of the protein to which it is conjugated. PEG and OEG linkers and related conjugation methods are described in, e.g., U.S. Pat. No. 6,716,821: U.S. Pat. No. 9,388,104: U.S. Patent Application Publication 2009/0285780; and Harris, J. M. and Chess, R. B., Nature Reviews Drug Discovery, 2:214-221 (2003).

The linker may comprise any suitable number of PEG or OEG molecules, units, or monomers. In some embodiments, the linker comprises 2-50 (e.g., 5, 10, 15, 20, 25, 30, 35, 40), or 45) PEG or OEG molecules. In other embodiments, the linker comprises 10-20 (e.g., 10, 11, 13, 14, 15, 16, 17, 18, 19, or 20) PEG or OEG molecules, or 30-40 (e.g., 31, 32, 33, 34, 35, 36, 37, 38, 39, or 40) PEG or OEG molecules. For example, the linker may comprise 16 OEG or PEG molecules, or 36 OEG or PEG molecules.

The ligand molecule disclosed herein further comprises a Z33 peptide of Staphylococcus aureus Protein A, or an antibody-binding fragment thereof, conjugated to the linker. Staphylococcal protein A (SPA) is a protein originally found in the cell wall of Staphylococcus aureus. It is composed of five homologous domains that fold into a three-helix bundle. Protein A plays an important role in immunology due to its specific binding to the Fc-portion of immunoglobulin G (IgG) from most mammalian species, including human. Extensive structural and biochemical studies of protein A have been conducted. The Z-58 domain of protein A was the first protein A domain widely used in affinity chromatography and affinity precipitation. The minimized binding domain Z-33 was later developed without significantly changing the function of the molecule. The Z33 peptide is a Protein A mimic having the amino acid sequence FNMQQQRRFYEALHDPNLNEEQRNAKIKSIRDD (SEQ ID NO: 1), and contains a motif of two α-helices. The disclosure also encompasses a ligand molecule comprising any antigen-binding fragment of a Z33 peptide, or a peptide having an amino acid sequence that is at least 95% (e.g., 95%, 96%, 97%, 98%, or 99%) identical to SEQ ID NO: 1. The degree of nucleic acid and/or amino acid identity can be determined using any method known in the art, such as the BLAST sequence database.

The disclosure further provides a method for purifying an antibody or an Fc fusion protein, which comprises (a) dissolving the above-described system in an aqueous solution at physiological pH and aging overnight, whereby the one or more filler molecules and one or more ligand molecules self-assemble into immunofibers (IFs); (b) mixing a sample containing a protein comprising an Fc region with the IFs under conditions whereby the IFs bind to the Fc region to form an immunofiber-protein complex in solution; (c) separating the immunofiber-protein complex from the solution by adding salt and/or centrifugation; and (d) dissociating the protein comprising an Fc region from the IFs. Any suitable protein that contains an Fc region may be purified using the methods disclosed herein. For example, the Fc region-containing protein may be an antibody, such as a monoclonal antibody. In other embodiments, the Fc region-containing protein may be an Fc fusion protein. An “Fc fusion protein” is a bioengineered polypeptide that joins the crystallizable fragment (Fc) domain of an antibody with another biologically active protein domain or peptide to generate a molecule with unique structure-function properties and, in some cases, therapeutic potential. The gamma immunoglobulin (IgG) isotype is often used as the basis for generating Fc-fusion proteins because of favorable characteristics such as recruitment of effector function and increased plasma half-life. In some embodiments, the system described herein may be used to capture and purify other proteins that do not comprise and Fc region by incorporating customized binding pairs into the design of the filler and ligand molecules.

“Physiologic” or “physiological” pH is the pH that typically occurs in human cells (e.g., human cells in vivo). In this regard, physiologic pH of the human body ranges between 7.35 to 7.45, with the average physiologic pH at 7.40. Once the filler molecules and ligand molecules are dissolved in the aqueous solution at the appropriate pH, the dissolved system is incubated or “aged” for a time sufficient for the one or more filler molecules and one or more ligand molecules to self-assemble into immunofibers (IFs). The incubation or “aging” of the filler and ligand molecules in solution may be for any suitable period of time. In some embodiments, the dissolved system is aged for at least two hours, but not more than 48 hours. For example, the dissolved system may be incubated or aged for 2-8 hours, 8-12 hours, 12-16 hours, 16-20 hours, 20-24 hours, 24-36 hours, or 36-48 hours. In some embodiments, the dissolved system is aged overnight (e.g., about 6, 7, 8, 9, 10, 11, or 12 hours).

Following a sufficient aging period and formation of IFs, the disclosed method comprises mixing a sample containing an antibody or an Fc fusion protein with the IFs under conditions whereby the IFs bind to the Fc region of the protein (e.g., an antibody or Fc fusion protein) to form an immunofiber-protein complex in solution. Suitable conditions for affinity-based antibody purification methods are known in the art and may be employed in the disclosed method. For example, reagents and conditions suitable for Protein A-based purification are known in the art and may be used in the context of the present disclosure (see, e.g., Proteus Protein A Antibody Purification Handbook, Bio-Rad (2016); and Fishman, J. B., and Berg, E. A., Cold Spring Harb Protoc: doi: 10.1101/pdb.prot099143). The complexes formed can then be separated from the unbound immunofibers and Fc region-containing proteins (e.g., antibodies or Fc fusion proteins) and other components in the sample by many known separation means, including, for example, salt-induced precipitation and centrifugation. The separated complexes can then be introduced into another solution at an acidic pH, where the immunofibers lose their binding affinity for the Fc region-containing proteins (e.g., antibodies or Fc fusion proteins).

The antibody or Fc fusion protein can then be dissociated from the immunofibers by filtration, such as diafiltration, microfiltration, or other means. In some embodiments, the antibody or Fc fusion protein is dissociated from the IFs by lowering the pH to elution conditions (e.g., pH 2.5-4.0) and filtration or microfiltration. In other embodiments, the antibody or Fc fusion protein may be dissociated from the IFs using sequential precipitation. In this regard, a sample containing an Fc region-containing protein (e.g., an antibody or an Fc fusion protein) may be mixed with a first IF solution (prepared as described above), aged or incubated for any suitable amount of time (e.g., 2, 3, 4, 5, or 6 hours), and centrifuged (e.g., at 10,000-20,000 rpm). The resulting supernatant, which contains immunofiber complexed with Fc region-containing protein, may then be mixed with a second IF solution (prepared as described above), aged or incubated for any suitable amount of time (e.g., 2, 3, 4, 5, or 6 hours), and centrifuged a second time (e.g., at 10,000-20,000 rpm). The aforementioned process may be repeated any number of times until a desired yield of Fc region-containing protein (e.g., antibody or Fc fusion protein) is reached, and the final precipitate is then washed and resuspended in elution buffer. In some embodiments, the eluted antibodies or Fc fusion proteins may be fully recovered using membrane separation methods.

The system and methods described herein have several advantages as compared to other protein purification methods known in the art, particularly protein A chromatography methods in which the capture step is one of the major downstream bottlenecks due to resin capacity limitation and high production cost. Indeed, the IF system and methods described herein provide for high throughput purification of monoclonal antibodies, ease of handling, and reduced cost. In addition, the disclosed system and methods allow for rapid purification of proteins, in that antibody-IF binding and agglomeration can be completed in 30 minutes or less.

The following examples further illustrate the invention but, of course, should not be construed as in any way limiting its scope.

### EXAMPLES

The following materials and methods were used in the experiments described in the Examples.

Materials. All Fmoc amino acids and resins were obtained from Advanced Automated Peptide Protein Technologies (AAPPTEC, Louisville, KY, USA). The pure mAb1, and mAb1 and mAb2 in clarified Chinese hamster ovary (CHO) cell culture harvest were obtained from Bristol-Myers Squibb (Devens, MA, USA). The cell culture was clarified using depth filtration. The Fmoc-N-amido-O(P) EGn-acids were purchased from PurePEG (San Diego, CA, USA) and BroadPharm (San Diego, CA, USA). Lauric acid (C12) was obtained from MilliporeSigma (St. Louis, MO, USA). Unless otherwise specified, all other reagents were obtained from VWR (Radnor, PA, USA) and used as received without further purification.

Molecular synthesis. Filler and ligand molecules were synthesized using the method described previously. In brief, C12-VVEE and C12-VVKKO(P)EGnGGZ33 (n=4, 8, 12, 16, 36, 45) were synthesized on the Liberty Blue automatic microwave peptide synthesizer (CEM Corporation, Matthews, NC, USA) using standard 9-fluorenyl-methoxycarbonyl (Fmoc) solid phase synthesis protocols. Crude products were cleaved from the solid support using a mixture of trifluoroacetic acid (TFA)/triisopropylsilane (TIS)/H₂O in a ratio of 92.5:5:2.5 for 2.5 hours. Excess TFA was removed by evaporation and ice-cold diethyl ether was added to precipitate the crude products, followed by centrifugation. The crude products were purified by preparative RP-HPLC using a Varian Polymeric Column (PLRP-S, 100 Å, 10 μm, 150×25 mm) at 25° C. on a Varian ProStar Model 325 preparative HPLC (Agilent Technologies, Santa Clara, CA, USA) monitored at 220 nm for the absorbance of peptide segments. Collected fractions were analyzed by MALDI-TOF (BrukerAutoflex III MALDI-TOF instrument, Billerica, MA, USA) and product-containing fractions were then lyophilized (LABCONCO™ FreeZone⁻105° C., 4.5 L freeze dryer, Kansas City, MO, USA) and stored at 30° C.

CMC measurement. The CMCs of the ligand molecules in PBS were determined using Nile Red, a hydrophobic dye that undergoes changes in both fluorescence intensity and emission wavelength (a blue-shift) upon partition into the hydrophobic domains of supramolecular assemblies. Nile Red was initially dissolved in acetone at 20 μM and 10 μl aliquots were loaded into several centrifuge tubes. After the acetone evaporated under room temperature. 500 μl fresh ligand solutions in PBS at various concentrations were added into the centrifuge tubes containing dry Nile Red and aged overnight for assembly. Fluorescent spectra of Nile Red were then monitored by a Fluorolog fluorometer (Jobin Yvon, Edison, NJ, USA) with fixed excitation wavelength at 560 nm; emission spectra were monitored at 580-720 nm. The ratio of the emission intensity at 635 nm (emission maximum of Nile red in a hydrophobic environment) to that at 660 nm (emission maximum of Nile red in an aqueous environment) was then plotted against the tested concentrations to obtain a transition curve, and the CMC value was determined by the intersection of the two fitting lines.

Self-Assembly, Co-Assembly, and TEM Imaging. For self-assembly, filler or ligand molecules were dissolved in PBS (20 mM sodium phosphate, 150 mM sodium chloride, pH 7.4) to reach a final concentration of 2.5 mM or 400 μM, respectively, and aged for 24 hours at room temperature. To construct co-assembled IFs, filler and ligand molecules were pretreated with hexafluoroisopropanol (HFIP) to eliminate any pre-existing nanostructures that could be possibly formed during the synthesis and purification process. After HFIP evaporation, filler and ligand molecules were dissolved in PBS (20 mM sodium phosphate, 150 mM sodium chloride, pH 7.4) to reach desired concentrations and aged for 24 hours at room temperature. 10 μl stock of each sample solution was then spotted on carbon film coated copper grids with 400 square mesh (Electron Microscopy Sciences, Hatfield, PA, USA) and the excess was removed with filter paper to leave a thin film of sample on the grid. After letting the sample dry for 5 minutes, 10 μl 2% uranyl acetate was added to the sample grid, and the excess was removed after 30 seconds. All samples were dried for at least three hours before imaging. Bright-field TEM imaging was performed on a FEI Tecnai 12 TWIN Transmission Electron Microscope and all TEM images were required by a SIS Megaview III wide-angle CCD camera.

CD Spectroscopy. The CD spectra of self-assembled ligand molecules were collected on a Jasco J-710 spectropolarimeter (JASCO, Easton, MD, USA) using a 1 mm path length quartz UV-vis absorption cell (ThermoFisher Scientific, Pittsburgh, PA, USA) at 25° C. A background spectrum of the solvent was acquired and subtracted from the sample spectrum. The collected data was averaged from three scans and normalized with respect to the ligand concentration.

ITC Experiment. ITC experiments were performed using a high precision VP-ITC titration calorimetric system (Microcal Inc.). 40 μM ligand solution was titrated with 100 μM mAb1 in PBS (pH 7.4) at 25° C. The heat evolved after each injection was obtained from the integral of the calorimetric signal. The heat associated with the binding between ligand molecule and mAb1 was obtained by subtracting the heat of dilution. Analysis of the data was performed using MicroCal Origin package.

mAb Precipitation Experiment. IF stock solutions at desired filler and ligand concentrations were prepared one day before the precipitation experiment. In experimental groups, pure mAb1 from a concentrated solution (448 μM, 64.5 g/L) was then added into 100 μL IF solutions to reach a desired final concentration and incubated for 30 minutes at room temperature. The solution was then centrifuged at 15000 rpm for 15 minutes. For the 1M salt group, ammonium sulfate was added to the mAb-IF mixtures to reach a final concentration of 1M and incubated for another 30 minutes before centrifugation. The supernatant was taken out and analyzed by ProA-HPLC (POROS™ A 20 μm Column, Stainless Steel, 2.1×30 mm, 0.1 mL) to determine the protein concentrations. The precipitated amount of each species was calculated by subtracting the amount in the supernatant from the amount added. The precipitation yield was calculated by dividing the added amount by the precipitated amount.

Sequential Precipitation and mAb Elution Using IF Solutions. Three IF stock solutions. IF1 (2.5 mM filler, 250 μM O16). IF2 (2.5 mM filler, 750 μM O16), and IF3 (2.5 mM filler. 200 μM O16), were prepared one day before the precipitation experiment. Pure mAb1 from a concentrated solution (448 μM, 64.5 g/L) was added into 100 uL IF1 and 100 uL IF2 to reach a desired final concentration of 40 μM and 80 μM, respectively. After a 4-hour incubation, the samples were then centrifuged at 15000 rpm for 15 minutes and the supernatant was transferred to 100 uL IF3 for another 4-hour incubation and subsequent centrifugation. 200 μL PBS and 400 μL elution buffer (40 mM sodium acetate. pH 3.7) were then used to wash and resuspend the precipitates from the two precipitation steps. The supernatant from each precipitation step, wash step, and elution step were analyzed by ProA-HPLC (POROS™ A 20 μm Column, Stainless Steel, 2.1×30 mm, 0.1 mL) to determine mAb concentration.

Purification of mAb1 from Clarified Cell Culture Harvest. mAb1 at clarified bulk state were incubated with optimized IFs (100 μL) at a concentration of 40 μM or 80 μM for 30 minutes. Same procedures were performed to precipitate mAb1 and obtain the centrifuged pellets. The pellets were then resuspended in PBS (400 μL, 40 mM sodium acetate. pH 3.7) and transferred to a dialysis tube (Pur-A-Lyzer Maxi, 50 kDa cut-off molecular weight. Sigma-Aldrich. St. Louis, MO. USA). The resuspended solution was then dialyzed against 40 mM sodium acetate (1 L) at pH 3.7 for 24 hours with dialysis buffer replaced three times. The protein, filler, and ligand concentrations were determined by ProA-HPLC and RP-UPLC.

Sequential Precipitation of High mAb Titers Using Lyophilized IFs. Six lyophilized IF stock powders (IF4-IF9) were prepared such that upon dissolution in 100 μL, the concentrations of the filler and O16 would be 2.5 mM and 400 μM, respectively. Lyophilized IF powders were prepared one day before the precipitation experiment. The IFs were allowed to co-assemble in water for 24 hours prior to lyophilization. Pure mAb1 from concentrated stock solutions of either (146 μM, 21 g/L) or (215 μM, 31 g/L) was added to the lyophilized IF4. After a 1-hour incubation, the samples were centrifuged at 15000 rpm for 15 minutes. Following centrifugation, the volume of supernatant was measured and fresh PBS was added until a final supernatant volume of 100 μL was reached. The 100 μL supernatant following the first precipitation step was then added to the next lyophilized IF stock powder (IF5) and allowed to incubate for another hour. This process of incubation, centrifugation, and supernatant transfer was repeated until all six lyophilized IFs (IF4-IF9) had been used for mAb precipitation. The supernatants after the first, third, and sixth precipitation steps were analyzed using ProA-HPLC to measure the cumulative amount of mAbs remaining throughout the precipitation process.

### Example 1

This example describes the molecular design and characterization of the system disclosed herein.

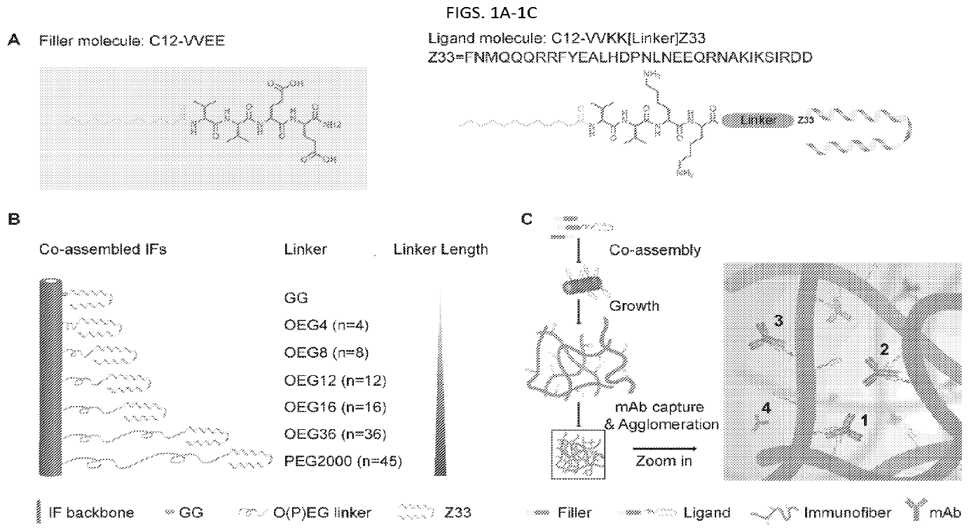

An immunofiber system was formed by the co-assembly of filler and ligand molecules. The filler molecule, C12-VVEE, was designed to modulate the distribution of the ligand molecule in the co-assembled IFs (FIG. 1A). The Z33 peptide, which possesses a two-helix motif, is conjugated on the C-terminus of the ligand molecule, C12-VVKK[Linker]Z33, for specific monoclonal antibody capture (FIG. 1A). In a previous ligand design (G2), a double glycine (GG) segment was inserted as a linker between the IF surface and Z33. To further increase Z33 flexibility and accessibility, a series of ligand molecules, O4, O8, O12, O16, O36, and P2000, with various OEG (or PEG) linkers (n=4, 8, 12, 16, 36, 45) were designed as summarized in Table 1.

TABLE 1
Summary of ligand molecule design with various linkers.

| Ligand Molecule | | Calculated Fully | Molecular | |
|---|---|---|---|---|
| C12- | | Extended Linker | Weight | CMC |
| VVKK[Linker]Z33 | Linker | Length (nm) | (kDa) | (μM) |
| G2 | GG | 0.7 | 4.9 | 2.0 |
| O4 | OEG4 | 1.9 | 5.0 | 5.1 |
| O8 | OEG8 | 3.5 | 5.2 | 6.0 |
| O12 | OEG12 | 5.1 | 5.3 | 8.5 |
| O16 | OEG16 | 6.7 | 5.5 | 10.5 |
| O36 | OEG36 | 14.6 | 6.4 | 9.1 |
| P2000 | PEG2000 | 18.2 | 6.8 | 8.1 |

As shown in FIG. 1B, with increasing OEG (or PEG) length, the Z33 peptide was expected to be extended further away from the IF surface. It was hypothesized that the incorporation of OEG (or PEG) linkers would further reduce the steric hindrance in the radial direction of the IFs and improve mAb-IF interactions for both Fc binding sites. The filler and ligand molecules were synthesized using automated solid-phase peptide synthesis (SPPS) methods.

FIG. 1C illustrates the spontaneous co-assembly process of filler and ligand molecules in aqueous solution, forming filamentous IFs with Z33 displayed on the IF surface. Upon the addition of mAbs, there were four possible states of mAbs in the IF solution. States 1 and 2 represent two Fc-portions of a mAb bound to two ligands from two different IFs or the same IF, respectively. In State 3, only one Fc-portion of a mAb is bound to one ligand on an IF. Given the equilibrium between ligand monomers and co-assembled structures, it was also possible that in State 4 mAbs are bound to zero, one, or two monomer ligands in the solution instead of those assembled in IFs. mAb binding and precipitation represent two separate processes. Previous results showed that 1 M salt was not able to precipitate mAb1 directly, but it served as a strong charge screening agent to precipitate almost all IFs and thus precipitate mAbs that are bound to IFs (State 1-3). Under no salt conditions, however, binding to IFs may not be sufficient for mAbs to be precipitated. The key to the precipitation of mAbs under no salt conditions may lie in the formation of the cross-linked mAb-IF complexes, which is mainly triggered by mAbs in State 1.

### Example 2

This example describes the molecular assembly and characterization of immunofibers produced using the system and methods disclosed herein.

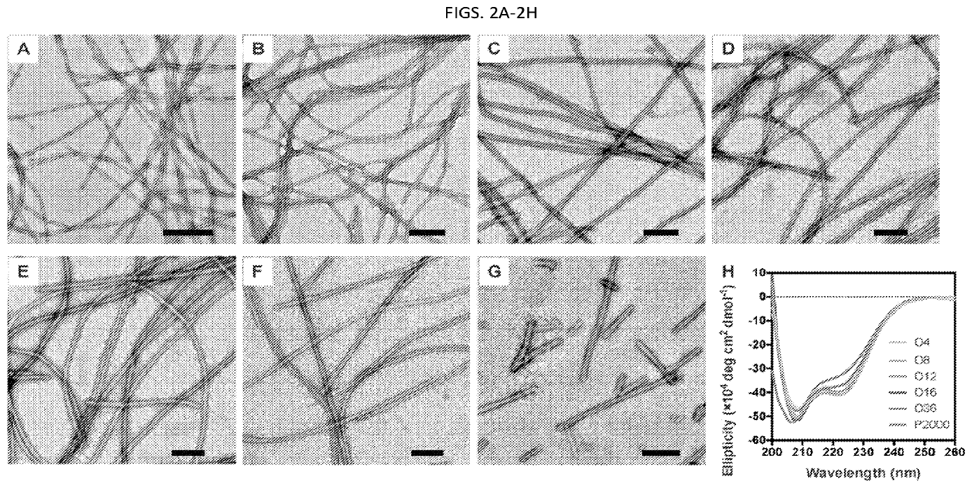

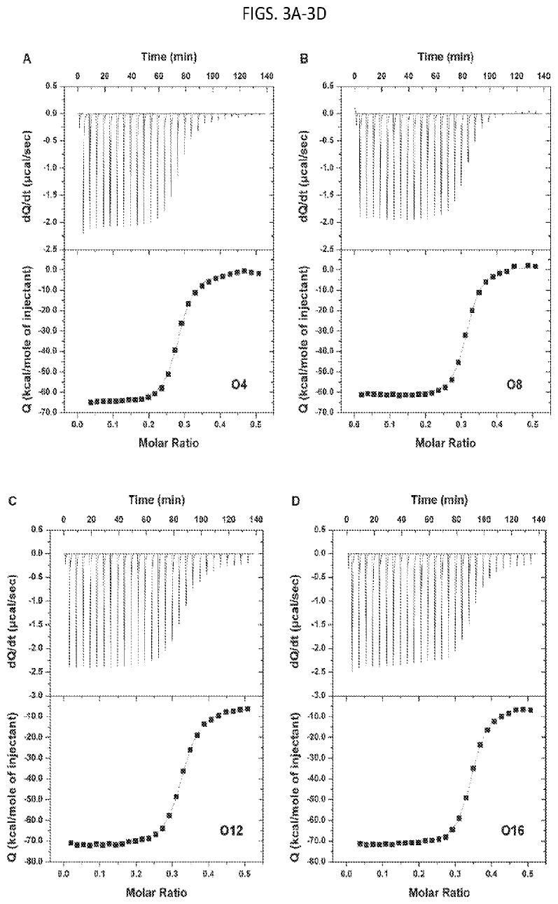

To estimate the behavior of the filler and ligand molecules in the co-assembled IFs, their self-assembly properties were first investigated in phosphate-buffered saline (PBS), pH 7.4. As shown in FIG. 2A, the filler molecule was able to self-assemble into filamentous structures with a diameter of 7.4±0.6 nm. With OEG (or PEG) linkers, the critical micelle concentrations (CMCs) of ligand molecules were relatively higher than the previous G2 ligand, ranging from 5.1 to 10.5 μM (Table 1). Representative transmission electron microscopy (TEM) images revealed that each of the ligand molecules can self-assemble into filamentous structures with diameters of 11.1±0.9 nm (O4), 12.6±1.2 nm (O8), 13.6±1.2 nm (O12), 14.9±1.3 nm (O16), 18.5±1.8 nm (O36), and 19.3±2.0 nm (P2000), respectively (FIGS. 2B-2G). As expected, the IF diameter increased with the increase of the linker length. To gain insight into the molecular packing within the self-assembled ligand molecules, the circular dichroism (CD) spectra were collected for each of the newly designed ligand molecules. The negative peaks around 208 nm and 222 nm suggest the preservation of α-helix secondary structures in all the self-assembled ligand molecules (FIG. 2H), as shown in the original Z33 peptide. Isothermal titration calorimetry (ITC) were used to determine the binding affinity between a monoclonal human IgG1 (144 kDa, abbreviated as “mAb1”) and ligand molecules 04-O16. The dissociation constant Kd ranged between 50 nM and 70 nM, similar to the reported value (26 nM) for free Z33 peptide, showing that the conjugation of OEG linkers within ligand molecules would not significantly impair their mAb binding affinity (FIGS. 3A-3D).

### Example 3

This example demonstrates the impact of linker length and ligand selection on antibody yield.

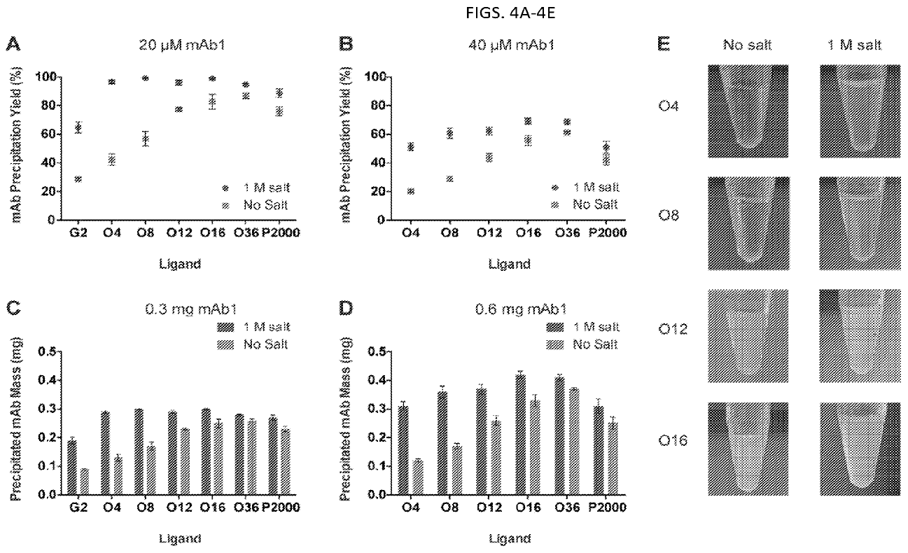

To compare the ligand performance. 100 μM ligand molecules were individually co-assembled with 2.5 mM filler molecules in 100 μL PBS (pH 7.4) using similar methods described previously. The filler molecule at 2.5 mM (solubility limit) was found to give the best mAb precipitation results and was consistently used in experiments described herein. Pure mAb1 from a stock solution was then added into different IF solutions to reach a final concentration of 20 μM and incubated for 30 minutes at room temperature. To investigate salt effects on the mAb precipitation yield, two parallel experiments were performed for each co-assembled IF system: one with 1 M salt and one without salt (FIG. 4A). After centrifugation, the supernatant was analyzed by ProA-HPLC to determine the remaining mAb concentration and calculate the mAb precipitation yield. With 1M salt, the mAb precipitation yield showed significant improvement from 65% to higher than 88% when replacing the GG linker with O(P)EG linkers in the ligand design. In particular, greater than 95% mAb precipitation yields were obtained for O4-O36. Since there is no bias against the IF precipitation efficiency under 1M salt conditions, this mAb precipitation yield increase from G2 to the new ligand molecules with OEG (or PEG) linkers specifically reflects the significant improvement in the mAb binding efficiency as a result of the increased linker length and flexibility. A slight drop in the mAb precipitation yield was seen for P2000, likely due to the entanglement of long PEG chains that undermines the accessibility of the Z33 peptide for mAb capture.

Under no salt conditions, the mAb precipitation yields were generally lower than salt groups, while an upward trend was observed with an increase of the OEG linker length. Unlike the salt group, the mAb precipitation yield under no salt conditions was determined by a combination of mAb binding efficiency and the precipitation efficiency of the mAb-IF complexes. From O4 to O36, there were no obvious differences in the mAb binding efficiency as indicated by the salt group data set. The increase in the mAb precipitation yield under no salt conditions represents an enhancement in the mAb precipitation efficiency, possibly caused by the improved crosslinking between IFs. More importantly, the yield difference between 1M salt and no salt groups was gradually diminished with increasing the linker length, leading to a less than 10% yield discrepancy for O36. This was considered very promising for mAb precipitation, in that the improved mAb-IF interactions achieved by using OEG or (PEG) linkers could potentially substitute for the salt contribution to mAb precipitation.

To confirm the above observation and have a preliminary understanding of the mAb binding capacity for this IF system, the experiments were repeated with a higher mAb1 concentration (40 μM). Similar yield trends were observed, as shown in FIG. 4B, except for a slightly upward trend in the 1M salt group for O4-O36, indicating an improvement in mAb binding efficiency with increasing the OEG linker length. Despite the consistent drop in the mAb precipitation yield from 20 to 40 μM mAbs, the precipitated mAb mass for a 100 μL sample (20 μM: 0.3 mg: 40 μM: 0.6 mg) were actually similar or higher for 40 μM mAbs (FIGS. 4C-4D), showing that the Z33 peptide displayed on the IF surfaces were not fully saturated by 20 μM mAb1 and could potentially be further utilized if more mAbs were added.

The difference in mAb precipitation yield can also be revealed by the cloudiness of the IF solution. IF solutions of O12, O16, O36, and P2000 became cloudy within 5 minutes after the mAb addition, which was not seen in the first three IF systems. FIG. 4E shows the sample vials of O4-O16 IFs after being mixed with 40 μM mAb1 for 5 minutes under 1 M salt or no salt conditions. Generally, the cloudiness of the salt groups was relatively higher than the no salt groups, suggesting a higher mAb precipitation yield for the salt groups. Meanwhile, the cloudiness increased from O4 to O16 in accordance with the yield trend shown in FIG. 4B.

These results demonstrate that an increase of the linker length can simultaneously improve the mAb binding efficiency of the ligand molecules as well as the precipitation efficiency of the mAb-IF complex. Although O36 showed the best mAb precipitation yields in all conditions, O16 is more desirable in terms of the synthesis yield and material cost and was selected for the subsequent experiments.

### Example 4

This example describes the optimization of monoclonal antibody precipitation under no salt conditions using the methods disclosed herein.

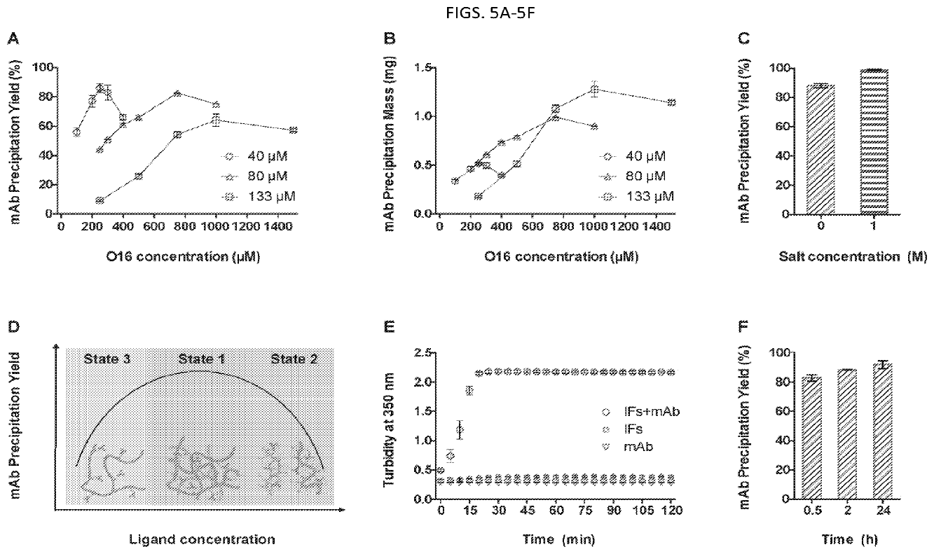

In previous studies, the ligand: mAb molar ratio was shown to play the most significant role in the mAb precipitation yield. To optimize the mAb precipitation yield under no salt conditions and investigate whether the O16 IF system could be efficiently applied to high mAb concentrations, mAb1 at three concentrations, 40 μM (6 mg/ml), 80 M (12 mg/ml), 133 μM (20 mg/ml), was incubated with IFs formed by 2.5 mM filler molecule and O16 ligand molecule at various concentrations in 100 μL PBS. FIGS. 5A and 5B show the plots of mAb precipitation yield and mass versus O16 concentration, respectively, and an optimal point was observed at a ligand:mAb molar ratio between 6:1 to 9:1 for all three mAb concentrations. At the optimal O16 concentration, the mAb precipitation yields for 40 and 80 μM mAb were around 85%, while only about 65% mAb was precipitated for 133 μM mAb, showing a decrease in the IF performance when scaling up for high mAb concentrations. To better understand why a 100% mAb precipitation yield was not reached at the optimal O16 concentration, parallel experiments were carried out for 40 μM mAb with the optimal IFs (2.5 mM filler, 250 μM O16) under both 1 M salt and no salt conditions (FIG. 5C). More than 99% mAb1 was precipitated when 1 M salt was present, indicating a complete capture of mAb1 onto IFs. However, only 88% mAb1 was precipitated for the no salt group, which was attributed to the insufficient IF precipitation that may be caused by the limited IF crosslinking.

To understand the trend for mAb precipitation yield observed in FIGS. 5A and 5B, States 3, State 1, and State 2 likely are dominant at low, medium, and high ligand concentrations, respectively (FIG. 5D). At low ligand concentration, all ligands are saturated by the excessive mAbs with little left for the binding of the second Fc site (State 3). The initial yield increase could be attributed to the increase in dual site mAb binding for IF crosslinking (State 1) as the ligand concentration increases. After the optimal operating point, further increasing ligand concentration resulted in a yield decline which could be caused by a decrease in the crosslinking efficiency, since chances for mAbs to bind to two Z33 from the same IF (State 2) would be much higher with decreased spacing of O16 ligands on IFs surfaces.

To gain insight into the kinetics of the agglomeration, the turbidity of a mixture of 40 μM mAb1 and the optimized IFs (2.5 mM filler, 250 μM O16) was monitored by the absorbance at 350 nm for 24 hours. As shown in FIG. 5E, the turbidity increased with time and plateaued within 30 minutes, suggesting rapid initial agglomeration. To correlate the solution turbidity to the mAb precipitation yield, the supernatant were analyzed for 30 minutes, 2 hours, and 24 hours (FIG. 5F). Consistent with the turbidity study, the mAb-IF binding and agglomeration could nearly complete within 30 minutes to reach a high precipitation yield of 83%. Although a slight yield increase was seen for 2 hours (88%) and 24 hours (92%), which was not revealed by the lower-sensitivity turbidity study, it sacrificed the time efficiency of the mAb precipitation process.

### Example 5

This example describes sequential precipitation and elution of monoclonal antibodies.

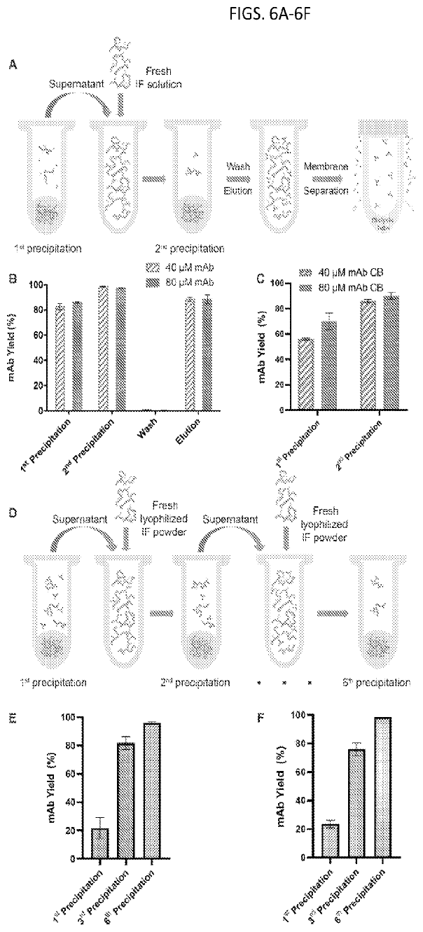

To further improve the mAb precipitation yield, a two-step sequential precipitation was conducted to precipitate the remaining mAbs. As depicted in FIG. 6A, the supernatant from the first precipitation step was added into fresh IF solution. A wash step was then carried out using PBS to remove the non-specifically bound impurities in the precipitates from the two precipitation steps. To resuspend the precipitated mAbs, elution buffer (40 mM sodium acetate, pH 3.7) was added to dissociate the mAb-IF complexes.

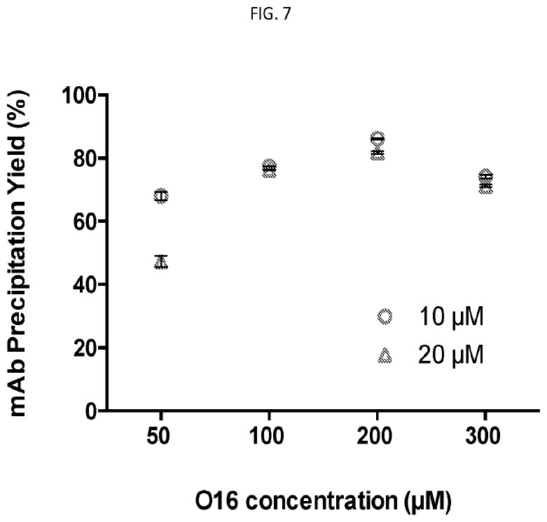

As a proof of concept, the sequential precipitation was carried out with pure mAb1 at 40 μM and 80 μM. For the first precipitation, 40 μM (6 mg/mL) and 80 μM (12 mg/mL) mAb1 were incubated with IFs containing 250 μM and 750 μM O16, respectively, the optimized condition indicated by FIG. 5A. More than 82% mAb1 was precipitated for both groups while 8˜20 μM mAb1 remained in the supernatant (FIG. 6B). Based on another set of O16 concentration optimizations shown FIG. 7, IFs containing 200 μM O16 was used for the second precipitation step. Eventually, a final precipitation yield of greater than 97% and ˜88% elution yield were achieved for both mAb concentrations with little yield loss during the wash step (FIG. 6B). To fully recover the eluted mAbs, a membrane separation step using a 50 kDa cut-off membrane will be conducted to separate the mAb1 (˜144 kDa) and dissociated IFs (monomeric size <6 kDa) (FIG. 6A). This sequential precipitation using dissolved IFs was then applied to the separation of mAb1 from clarified cell culture harvest at 40 μM and 80 μM, where more than 86% and 90% of mAb1 precipitation yield was achieved for the respective mAb titers (FIG. 6C).

To achieve high mAb precipitation yields at mAb titers of over 20 mg/mL, sequential precipitation was carried out using fresh lyophilized IFs containing 2.5 mM filler and 400 μM O16 mixed with 100 μL of either 21 mg/mL or 31 mg/mL pure mAb. The mAbs were incubated with lyophilized IFs for 1 hour prior to centrifugation. Following the centrifugation step, the supernatant, still containing mAbs, was measured and fresh PBS was added until a final volume of 100 μL was reached. The precipitation step was repeated with freshly prepared lyophilized IFs at the same concentration until a total of six precipitation steps had been completed (FIG. 6D). The supernatants after the first, third, and sixth precipitation steps were analyzed using ProA-HPLC to measure the cumulative amount of mAbs remaining throughout the precipitation process. As shown in FIGS. 6E and 6F, nearly all the mAbs were precipitated after six incubation and centrifugation steps, with yields reaching 96% and 98% for 21 mg/mL and 31 mg/ml mAb titers, respectively. Furthermore, after only three precipitation steps, mAb precipitation yields increased drastically from around 20% to 80%, indicating that optimal ligand:protein ratios are reached early in the process as more proteins are captured and separated with each successive precipitation step.

The above Examples illustrate the design and construction of a series of supramolecular IF systems containing OEG (or PEG) linkers and demonstrate the impact of epitope topography in the radial direction of IFs on the IF bioactivity. The described results reveal that increasing the linker length to OEG16 can simultaneously improve monoclonal binding and precipitation efficiency under no salt conditions. Linkers that are too long, however, show an adverse impact on the function of the resultant supramolecular polymers. Importantly, the mAb precipitation yield under no salt conditions may be efficiently optimized by tuning the ligand concentration to reach desired monoclonal antibody binding states. The strategy of engineering linkers for better epitope presentation sheds important light on the design of supramolecular polymers for specific molecular recognition and targeted drug delivery. The supramolecular IF system described herein can serve as an efficient alternative for the purification of monoclonal antibodies, and may be applied to the capture and purification of other molecules of interest by incorporating customized binding pairs into the system design.

### REFERENCES

All references, including publications, patent applications, and patents, cited herein are hereby incorporated by reference to the same extent as if each reference were individually and specifically indicated to be incorporated by reference and were set forth in its entirety herein.
1. Adelizzi, B.; Van Zee, N. J.; de Windt, L. N. J.; Palmans, A. R. A.; Meijer, E. W. Future of Supramolecular Copolymers Unveiled by Reflecting on Covalent Copolymerization. Journal of the American Chemical Society 2019, 141, 6110-6121.
2. Aida, T.; Meijer, E. W. Supramolecular Polymers-We've Come Full Circle. Israel Journal of Chemistry 2020, 60, 33-47.
3. Stupp, S. I.; Clemons, T. D.; Carrow, J. K.; Sai, H.; Palmer, L. C. Supramolecular and Hybrid Bonding Polymers. Israel Journal of Chemistry 2020, 60, 124-131.
4. Webber, M. J.; Appel, E. A.; Meijer, E. W.; Langer, R. Supramolecular Biomaterials. Nature Materials 2016, 15, 13-26.
5. Shy, A. N.; Kim, B. J.; Xu, B. Enzymatic Noncovalent Synthesis of Supramolecular Soft Matter for Biomedical Applications. Matter 2019, 1, 1127-1147.
6. Aida, T.; Meijer, E. W.; Stupp, S. I. Functional Supramolecular Polymers. Science 2012, 335, 813-817.
7. Sato, K.; Hendricks, M. P.; Palmer, L. C.; Stupp, S. I. Peptide Supramolecular Materials for Therapeutics. Chemical Society Reviews 2018, 47, 7539-7551.
8. Cui, H. G.; Webber, M. J.; Stupp, S. I. Self-Assembly of Peptide Amphiphiles: From Molecules to Nanostructures to Biomaterials. Biopolymers 2010, 94, 1-18.
9. Acar, H.; Srivastava, S.; Chung, E. J.; Schnorenberg, M. R.; Barrett, J. C.; LaBelle, J. L.; Tirrell, M. Self-Assembling Peptide-Based Building Blocks in Medical Applications. Advanced Drug Delivery Reviews 2017, 110, 65-79.
10. Su, H.; Wang, F. H.; Ran, W.; Zhang, W. J.; Dai, W. B.; Wang, H.; Anderson, C. F.; Wang, Z. Y.; Zheng, C.; Zhang, P. C.; Li, Y. P.; Cui, H. G. The Role of Critical Micellization Concentration in Efficacy and Toxicity of Supramolecular Polymers. Proceedings of the National Academy of Sciences of the United States of America 2020, 117, 4518-4526.
11. Wang, H. M.; Feng, Z. Q. Q.; Xu, B. Supramolecular Assemblies of Peptides or Nucleopeptides for Gene Delivery. Theranostics 2019, 9, 3213-3222.
12. Moore, A. N.; Hartgerink, J. D. Self-Assembling Multidomain Peptide Nanofibers for Delivery of Bioactive Molecules and Tissue Regeneration. Accounts of Chemical Research 2017, 50, 714-722.
13. VandenBerg, M. A.; Sahoo, J. K.; Zou, L.; McCarthy. W.; Webber, M. J. Divergent Self-Assembly Pathways to Hierarchically Organized Networks of Isopeptide-Modified Discotics under Kinetic Control. ACS Nano 2020.
14. Wang, F. H.; Xu, D. Q.; Su, H.; Zhang, W. J.; Sun, X. R.; Monroe, M. K.; Chakroun, R. W.; Wang, Z. Y.; Dai, W. B.; Oh, R.; Wang, H.; Fan, Q.; Wan, F. Y.; Cui, H. G. Supramolecular Prodrug Hydrogelator as an Immune Booster for Checkpoint Blocker-Based Immunotherapy. Science Advances 2020, 6.
15. Chen, C. H.; Palmer, L. C.; Stupp, S. I. Self-Repair of Structure and Bioactivity in a Supramolecular Nanostructure. Nano Letters 2018, 18, 6832-6841.
16. Storrie, H.; Guler, M. O.; Abu-Amara, S. N.; Volberg, T.; Rao, M.; Geiger. B.; Stupp, S. I. Supramolecular Crafting of Cell Adhesion. Biomaterials 2007, 28, 4608-4618.
17. Du, X. W.; Zhou. J.; Xu, B. Supramolecular Hydrogels Made of Basic Biological Building Blocks. Chemistry-an Asian Journal 2014, 9, 1446-1472.
18. Sur, S.; Tantakitti, F.; Matson, J. B.; Stupp, S. I. Epitope Topography Controls Bioactivity in Supramolecular Nanofibers. Biomaterials Science 2015, 3, 520-532.
19. Pashuck, E. T.; Duchet, B. J. R.; Hansel, C. S.; Maynard, S. A.; Chow, L. W.; Stevens, M. M. Controlled Sub-Nanometer Epitope Spacing in a Three-Dimensional Self-Assembled Peptide Hydrogel. ACS Nano 2016, 10, 11096-11104.
20). Guler, M. O.; Hsu, L.; Soukasene, S.; Harrington, D. A.; Hulvat. J. F.; Stupp, S. I. Presentation of Rgds Epitopes on Self-Assembled Nanofibers of Branched Peptide Amphiphiles. Biomacromolecules 2006, 7, 1855-1863.
21. Hudalla, G. A.; Sun, T.; Gasiorowski, J. Z.; Han, H. F.; Tian, Y. F.; Chong, A. S.; Collier, J. H. Gradated Assembly of Multiple Proteins into Supramolecular Nanomaterials. Nat. Mater. 2014, 13, 829-836.
22. Cheetham, A. G.; Zhang, P. C.; Lin, Y. A.; Lock, L. L.; Cui, H. G. Supramolecular Nanostructures Formed by Anticancer Drug Assembly. Journal of the American Chemical Society 2013, 135, 2907-2910.
23. Trent, A.; Marullo, R.; Lin, B.; Black, M.; Tirrell, M. Structural Properties of Soluble Peptide Amphiphile Micelles. Soft Matter 2011, 7, 9572-9582.
24. Bolton, G. R.; Mehta, K. K. The Role of More Than 40 Years of Improvement in Protein a Chromatography in the Growth of the Therapeutic Antibody Industry. Biotechnol. Prog. 2016, 32, 1193-1202.
25. Gronemeyer, P.; Ditz, R.; Strube, J. Trends in Upstream and Downstream Process Development for Antibody Manufacturing. Bioengineering 2014, 1, 188-212.
26. Mullerpatan, A.; Chandra, D.; Kane, E.; Karande, P.; Cramer, S. Purification of Proteins Using Peptide-Elp Based Affinity Precipitation. Journal of Biotechnology 2020, 309, 59-67.
27. Venkiteshwaran, A.; Heider, P.; Teyssevre, L.; Belfort, G. Selective Precipitation-Assisted Recovery of Immunoglobulins from Bovine Serum Using Controlled-Fouling Crossflow Membrane Microfiltration. Biotechnology and Bioengineering 2008, 101, 957-966.
28. Fausnaugh, J. L.; Pfannkoch, E.; Gupta, S.; Regnier, F. E. High-Performance Hydrophobic Interaction Chromatography of Proteins. Anal Biochem 1984, 137, 464-72.
29. Baldwin, R. L. How Hofmeister Ion Interactions Affect Protein Stability. Biophysical Journal 1996, 71, 2056-2063.
30). Majumdar, R.; Manikwar, P.; Hickey, J. M.; Samra, H. S.; Sathish, H. A.; Bishop, S. M.; Middaugh, C. R.; Volkin, D. B.; Weis, D. D. Effects of Salts from the Hofmeister Series on the Conformational Stability. Aggregation Propensity, and Local Flexibility of an Igg1 Monoclonal Antibody. Biochemistry 2013, 52, 3376-3389.
31. Swartz, A. R.; Xu, X. K.; Traylor, S. J.; Li, Z. J.; Chen, W. One-Step Affinity Capture and Precipitation for Improved Purification of an Industrial Monoclonal Antibody Using Z-Elp Functionalized Nanocages. Biotechnology and Bioengineering 2018, 115, 423-432.
32. Li, Y.; Lock, L. L.; Wang, Y. Z.; Ou, S. H.; Stern, D.; Schon, A.; Freire, E.; Xu, X. K.; Ghose, S.; Li, Z. J.; Cui, H. G. Bioinspired Supramolecular Engineering of Self-Assembling Immunofibers for High Affinity Binding of Immunoglobulin G. Biomaterials 2018, 178, 448-457.
33. Braisted, A. C.; Wells, J. A. Minimizing a Binding Domain from Protein A. Proc. Natl. Acad. Sci. U.S.A. 1996, 93, 5688-5692

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A system comprising:
(a) one or more filler molecules comprising a linear hydrocarbon chain conjugated to an amino acid sequence of 2-5 amino acids;
(b) one or more ligand molecules comprising a linear hydrocarbon chain, an amino acid sequence of 2-5 amino acids, a linker, and a Z33 peptide of Staphylococcus aureus Protein A, or an antibody-binding fragment thereof, conjugated to the linker, wherein the one or more filler molecules and one or more ligand molecules have the ability to self-assemble into immunofibers (IFs) under physiological conditions.

2. The system of claim 1, wherein the Z33 peptide comprises the amino acid sequence FNMQQQRRFYEALHDPNLNEEQRNAKIKSIRDD (SEQ ID NO: 1).

3. The system of claim 1 or claim 2, wherein each of the one or more filler molecules and one or more ligand molecules comprises a linear hydrocarbon chain of 12 carbons in length.

4. The system of any one of claims 1-3, wherein each of the one or more filler molecules and the one or more ligand molecules comprises an amino acid sequence of VVXX (SEQ ID NO: 2).

5. The system of claim 4, wherein the filler molecule comprises the amino acid sequence VVEE (SEQ ID NO: 3).

6. The system of claim 4, wherein the ligand molecule comprises the amino acid sequence VVKK (SEQ ID NO: 4).

7. The system of any one of claims 1-6, wherein the linker comprises one or more oligo (ethylene glycol) (OEG) molecules.

8. The system of any one of claims 1-6, wherein the linker comprises one or more polyethylene glycol (PEG) molecules.

9. The system of claim 7 or claim 8, wherein the linker comprises 2-50 OEG or PEG molecules.

10. The system of claim 9, wherein the linker comprises 36 OEG or PEG molecules.

11. The system of claim 9, wherein the linker comprises 16 OEG or PEG molecules.

12. A method for purifying a protein comprising an Fc region of an antibody, which comprises
(a) dissolving the system of any one of claims 1-11 in an aqueous solution at physiological pH and aging overnight, whereby the one or more filler molecules and one or more ligand molecules self-assemble into immunofibers (IFs);
(b) mixing a sample containing a protein comprising an Fc region with the IFs under conditions whereby the IFs bind to the Fc region to form an immunofiber-protein complex in solution;
(c) separating the immunofiber-protein complex from the solution by adding salt and/or centrifugation; and
(d) dissociating the protein comprising an Fc region from the IFs.

13. The method of claim 12, wherein the protein comprising an Fc region is an antibody.

14. The method of claim 12, wherein the protein comprising an Fc region is an Fc fusion protein

15. The method of any one of claims 12-14, wherein the protein comprising an Fc region is dissociated from the IFs by lowering the pH to elution condition and filtration or microfiltration.

16. The method of any one of claims 12-15, which is completed in 30 minutes or less.
